(19) United States
(12) Patent Application Publication
(10) Pub. No.: US 2011/0046497 A1
(43) Pub. Date: Feb. 24, 2011

(54) SENSOR PLATFORM FOR RESPIRATORY GAS ANALYSIS

(76) Inventors: Klaus Abraham-Fuchs, Erlangen (DE); Maximilian Fleischer, Hohenkirchen (DE); Karsten Hiltawsky, Schwerte (DE); Oliver Hornung, Furth (DE); Thomas Krüger-Sundhaus, Pommersfelden (DE); Erhard Magori, Feldkirchen (DE); Peter Paulicka, Rottenbach (DE); Roland Pohle, Herdweg (DE); Oliver von Sicard, Munchen (DE)

Correspondence Address: HARNESS, DICKEY & PIERCE, P.L.C., P.O.BOX 8910, RESTON, VA, 20195, US

(21) Appl. No.: 12/859,388

(22) Filed: Aug. 19, 2010

(30) Foreign Application Priority Data
Aug. 20, 2009 (DE) 10 2009 038 238.0

(51) Int. Cl. A61B 5/097

(52) U.S. Cl. 600/532

(57) ABSTRACT

An appliance is disclosed for measuring at least one gas analyte in exhaled air. In at least one embodiment, the appliance includes an inlet opening for the introduction of exhaled air (mouthpiece), at least one measuring chamber for receiving a sensor unit, and a conduit which provides a fluid connection from the opening to the measuring chamber, and wherein, depending on the gas analyte that is to be measured, a sensor unit with a suitable gas sensor can be introduced into the receiver.

### PRIORITY STATEMENT

The present application hereby claims priority under 35 U.S.C. §119 on German patent application number DE 10 2009 038 238.0 filed Aug. 20, 2009, the entire contents of which are hereby incorporated herein by reference.

### FIELD

At least one embodiment of the present invention generally relates to an arrangement for measuring analytes in respiratory gas and/or to a method for measuring the concentration of NO.

### BACKGROUND

Respiratory gas analyzers for medical diagnosis, monitoring of treatment or lifestyle applications are appearing in increasing numbers on the market. In particular, the development of inexpensive, selective and highly sensitive sensors will make it possible in future to develop small, portable and inexpensive appliances. However, development work has hitherto been undertaken separately for each use, e.g. asthma monitoring and blood alcohol.

There are many biomarkers, metabolic products and other substances that can be measured in respiratory gas and that can yield information concerning inflammatory diseases, cancers, metabolic disorders, or signs of poisoning or intoxication presented by the patient (see, for example, Pleil in J Toxicol Environ Health B Crit Rev. 2008 October; 11(8): 613-29. Role of exhaled breath biomarkers in environmental health science or Buszewski et al., Human exhaled air analytics: biomarkers of diseases, Biomed Chromatogr. 2007 June; 21(6): 553-66 Review). Respiratory gas analysis is already used specifically in the diagnosis of poisoning, asthma, diabetes, lung cancer, inflammatory diseases of the airways, and kidney or liver failure.

Hitherto, respiratory gas analysis directed at a wide range of target gases has been carried out only on very large and expensive apparatus, for example a mass spectrometer or gas chromatograph, especially for medical research. Small portable appliances in the mid-price range have hitherto been available on the market only for individual niche applications, for example NO determination for asthma monitoring or alcohol measurements. Moreover, “electronic noses” have been developed which integrate several sensors in one appliance in order to be able to identify complex odors by measuring techniques. However, arrays of nonspecific sensors are used for electronic noses, not highly selective sensors for individual target gases.

At the same time, increasingly smaller and more powerful sensors are being developed, e.g. FET sensors for detection of NO, IR sensors, electrochemical sensors, etc.

The market potential for respiratory gas analysis could be expanded considerably if a modular platform technology were made available which, on the basis of the same equipment, would permit any desired combination of target gases from a wide range of options.

### SUMMARY

In at least one embodiment of the invention, it is proposed that a modular base platform should integrate all the functionalities for respiratory gas analysis that are common to all possible measurement applications.

At least one embodiment of the invention covers in particular the subject matter of at least one of the following numbered paragraphs:

1. An appliance for measuring at least one gas analyte in exhaled air, said appliance having an inlet opening for the introduction of exhaled air, at least one measuring chamber for receiving a sensor unit, and a conduit which provides a fluid connection from the opening to the measuring chamber, and wherein, depending on the gas analyte that is to be measured, a sensor unit with a suitable gas sensor can be introduced into the receiver.
2. The appliance as in paragraph 1, having a plurality of measuring chambers.
3. The appliance as in paragraph 1 or 2, further having a valve for selectively connecting one measuring chamber, or a subgroup of measuring chambers from the total number of measuring chambers, to the inlet opening.
4. The appliance as in one of the preceding paragraphs, further having at least one gas-conditioning device.
5. The appliance as in one of the preceding paragraphs, having a partitioning device for partitioning the incoming exhaled air and, optionally, for delivering a predetermined portion to the measuring chamber or for delivering a predetermined portion to a predetermined measuring chamber of a plurality of measuring chambers.
6. The appliance as in one of the preceding paragraphs, further having a device for delivering a defined volume of exhaled gas to a defined measuring chamber.
7. The appliance as in one of the preceding paragraphs, further having a particle filter.
8. The appliance as in one of the preceding paragraphs, further having a one-way valve, such that air exhaled into the appliance can no longer be sucked out and re-inhaled by a user.
9. The appliance as in one of the preceding paragraphs, wherein the at least one gas analyte is nitrogen monoxide, and the device for gas conditioning is a device for oxidation of nitrogen monoxide to nitrogen dioxide.
10. The appliance as in paragraph 9, wherein the gas sensor is chosen from the group comprising gas-sensitive FET sensor, IR sensor, metal oxide sensor.
11. The appliance as in one of the preceding paragraphs, having a calibration gas device for supplying the measuring chamber with at least one calibration gas.
12. The appliance as in one of the preceding paragraphs, having a temperature control device for controlling the temperature of at least one measuring chamber.
13. The appliance as in one of the preceding paragraphs, wherein at least one measuring chamber is thermally insulated.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The measuring chamber forms a receiver in which the sensor unit can be received, such that a fluid connection from the conduit to the gas sensor is ensured.

There are preferably 2, 3, 4, 5, 6, 7, 8, 9 or 10 measuring chambers. According to an alternative embodiment, a measuring chamber has a plurality of receivers for a plurality of sensor units.

In the case of several measuring chambers, delivery conduits of different effective diameter can be provided, such that defined measuring chambers receive defined portions of the gas flow.

A gas conditioning device can be a device for dehumidification of air, for filtering of respiratory aerosols, an oxidation device, a heater, a filter or the like, so as to condition the exhaled air upstream of a measuring chamber (or of the one measuring chamber) for the measurement. The gas-conditioning device can likewise be fitted in a modular fashion in a receiver provided for the purpose. In this way, it can be easily exchanged, for example when used up, or the system can be equipped with one or more different gas-conditioning devices depending on the measurement requirements. The gas-conditioning device is arranged upstream from the measuring chamber.

A partitioning device can be embodied using simple mechanical elements or also using a control system (e.g. an electronic control system) with time-dependent or volume-dependent partitioning of the exhaled gas flow. For example, a branched conduit system can be provided for this purpose, in which a defined portion can be conveyed via a three-way valve or multi-way valve through a respective conduit branch to the corresponding measuring chamber.

The device for delivering a defined volume of exhaled gas can be embodied using simple mechanical elements or also using a control system (e.g. an electronic control system).

The sensor unit can preferably be inserted into the measuring chamber from the outside, without the respiration appliance having to be opened. Particularly preferably, the sensor unit can be inserted or replaced without additional tools, e.g. using a plug connection.

By way of a thermal insulation of the measuring chamber, several gas sensors can each be operated at their optimum temperature, without influencing one another.

An appliance according to at least one embodiment of the invention can further comprise the following features:
a mouthpiece and/or gas flow channels for taking a sample from the respiratory gas
a processor, e.g. for measurement control, for recording measured values and/or for data processing
valves and pumps for controlling the flow of gas and for flushing out the measurement gas (e.g. with the aid of ambient air)
if appropriate, reference gas devices for calibration, e.g. in the form of a gas reservoir or gas generator
filters for cleaning and dehumidifying the respiratory gas
filters for cleaning the flushing gas
devices for quality control of the measurement, e.g. volume control, humidity control, temperature control and the like
data output device, e.g. a display.

The calibration gas can be purified air that is produced, for example, by outside air being sucked in through a cleaning gas filter. The latter can contain activated charcoal, for example.

This modular measurement platform is configured in such a way that, without further outlay in terms of construction, any desired choice of gas sensors from a predetermined range of target gases can be integrated in the same appliance. Typically, sensors for 2 to 4 target gases are mounted together in the modular platform.

During the period when the appliance is not in use, the measuring chamber can preferably be closed off in a gas-tight manner by valves, such that no ambient air can reach the sensor during this period.

In the case of a plurality of measuring chambers and a branched system of conduits, it is left to the person skilled in the art to decide on the specific arrangement of gas-conditioning devices, valves, partitioning devices, and devices for measuring a volume of respiratory gas.

A typical choice of gas analytes is, for example:
NO for asthma monitoring
CO₂ for fertility diagnosis
H₂ for diagnosis of a Heliobacter pylori infection
carbon monoxide (CO) for diagnosis of inflammatory diseases of the airways
volatile organic compounds, e.g. alcohols, aldehydes, carboxylic acids or ketones, e.g. alcohol for determining the blood alcohol level from the respiratory gas, or, for example, acetone for optimizing fitness training.

This permits the combination of 2 or more sensors of the same type or of different types (e.g. gas FET, IR sensor, metal oxide sensor) in one appliance, as a result of which the appliance can be adapted to the particular requirements.

A valve control system is preferably provided which conveys the targeted choice of a defined portion of the exhaled gas flow (e.g. only end-expiratory portion or entire gas flow) through the sensors, it being possible for the partitioning to be different for each target gas.

The basic appliance is of a modular construction, such that it can be equipped with different sensors for different target gases, and for example can be adapted to the different measuring tasks by means of software in a control processor.

The appliance preferably has a gas outlet which, when a measurement procedure continues after the introduction of air, prevents outside air from entering the measuring chamber and distorting the measurement.

The appliance preferably comprises one or more valves in order to close the measuring chamber in a gas-tight manner during the measurement procedure.

The measuring chambers can be thermally insulated in such a way that they can be operated at elevated temperatures, in order to avoid undesired adherence of gas molecules to the wall of the measuring chamber. The gas measurement can also be carried out, using sensors with different temperature control, with local temperature differences of up to several hundred degrees. The sensors can be accommodated in a common measuring chamber or in different measuring chambers.

If the appliance has two or more measuring chambers, the incoming flow of gas is divided up through a suitable branch system upstream of the measuring chambers, and the subsidiary amounts are each delivered to different measuring chambers. A gas converter (e.g. for conversion of NO to NO₂) can in this case be fitted only in one of the several delivery paths.

One or more of the sensors can be used for highly specific measurement of gases other than the target gases, in which case the sensors for the target gases have cross-sensitivities to the other gases, and the additional measured values from the highly specific other sensors are used to correct the measured values of the target gas sensors.

The modular measurement platform for respiratory gas analysis, as proposed according to the invention, affords an increased market potential for respiratory gas analysis, since the development cost for new respiratory gas analysis devices is greatly reduced, and the appliances can therefore be produced much less expensively.

Inexpensive NO sensors with the required sensitivity in the ppb range have not hitherto been available on the market. A newly developed NO₂ sensor on the basis of Suspended Gate FET technology meets the stated requirements.

However, a gas-conditioning device for converting the NO in the respiratory gas to NO₂, which can be detected by the sensor, has to be provided upstream of such a sensor. The gas-conditioning device should ideally last for several months or even for several years, should be inexpensive and should convert NO to NO₂ at a constant conversion rate that is as high as possible.

The conversion of nitrogen monoxide to nitrogen dioxide takes place according to the following reaction equation:

2NO+O₂ [symbol] 2NO₂

The conversion of nitrogen monoxide to nitrogen dioxide can take place, in a respiratory gas sensor appliance, by way of a gas-conditioning device for oxidation of nitrogen monoxide to nitrogen dioxide, e.g. by conveying the (respiratory) air through an oxidation agent (e.g. potassium permanganate) or an oxidation catalyst that uses atmospheric oxygen or residual oxygen of the respiratory air for the oxidation.

A further problem is the fact that NO₂ dissolves in water much better than NO does. A method is therefore needed by which the concentration of the converted NO₂ in the moist respiratory gas can be measured constantly and quantitatively. Because of the greater solubility of NO₂ in water, some of the (converted) NO₂ in (respiratory) air with a high moisture content is dissolved in water, the concentration of the measurable NO₂ drops, and an NO content is measured that is apparently too low.

To permit a quantitative measurement of the NO content, a further gas-conditioning device, for example, should be chosen in order to dehumidify the air, and then a device for oxidation of nitrogen monoxide to nitrogen dioxide.

The appliance according to an embodiment of the invention in this respect offers the possibility of providing measuring chambers for several sensors and of providing further receivers for several gas-conditioning devices, such that the appliance, as in the above example, can in each case be adapted to the measurement application.

The mouthpiece, the gas-conditioning device and the sensor unit can be designed as disposable articles, which can be replaced after a single use or after a limited number of uses.

Depending on the application, the filling of the measuring chamber can take place much more quickly than the actual measuring procedure of the sensor. Here, care must be taken to ensure that, while the measurement is still ongoing, no outside air enters the measuring chamber and thus distorts the measurement. During the measurement procedure, therefore, the measuring chamber must in this case be closed. This can be done by using a delivery and removal conduit which leads to and from the measuring chamber and has a sufficiently high resistance to air movement. Alternatively, the measuring chamber can be closed off by one or two valves during the measurement procedure.

In the rest phase, some of the sensors that are of interest and can be used for said purposes can react with constituents of the ambient air, which causes distortion of the sensor display, e.g. in the form of a change in the sensor zero point. This is advantageously avoided by providing devices such as valves which, during the period when the appliance is not in use, suppress the admission of outside air to sensors located in the measuring chamber.

Important advantages of the overall system lie in the use of a noninvasive measuring method. The measurements can be repeated in large numbers and can thus also be used for monitoring the course of treatments, in the diagnosis of various diseases, etc. The system presented here is therefore also suitable for use outside of hospitals and medical practices.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An appliance for measuring at least one gas analyte in exhaled air, said appliance comprising:
an inlet opening for the introduction of exhaled air;
at least one measuring chamber for receiving at least one sensor unit; and
a conduit to provide a fluid connection from the inlet opening to the measuring chamber, wherein, depending on the gas analyte that is to be measured, a sensor unit with a gas sensor is introduceable into the receiver.

2. The appliance as claimed in claim 1, wherein the at least one measuring chamber includes a plurality of measuring chambers.

3. The appliance as claimed in claim 1, further comprising a valve for selectively connecting the at least one measuring chamber to the inlet opening.

4. The appliance as claimed in claim 1, further comprising at least one gas-conditioning device.

5. The appliance as claimed in claim 1, further comprising a partitioning device for partitioning the incoming exhaled air and, optionally, for delivering a portion to the at least one measuring chamber.

6. The appliance as claimed in claim 1, further comprising a device for delivering a defined volume of the exhaled gas to a defined measuring chamber.

7. The appliance as claimed in claim 1, further comprising a particle filter.

8. The appliance as claimed in claim 1, further comprising:
a one-way valve, to prevent air exhaled into the appliance from being sucked out and re-inhaled by a user.

9. The appliance as claimed in claim 4, wherein the at least one gas analyte is nitrogen monoxide, and wherein the device for gas conditioning is a device for oxidation of nitrogen monoxide to nitrogen dioxide.

10. The appliance as claimed in claim 9, wherein the gas sensor is chosen from the group comprising NO₂-sensitive FET sensor, IR sensor, metal oxide sensor.

11. The appliance as claimed in claim 1, further comprising:
a calibration gas device for supplying the measuring chamber with at least one calibration gas.

12. The appliance as claimed in claim 1, further comprising:
a temperature control device for controlling the temperature of at least one measuring chamber.

13. The appliance as claimed in claim 1, wherein at least one measuring chamber is thermally insulated.

14. The appliance as claimed in claim 2, further comprising a valve for selectively connecting a subgroup of the plurality of measuring chambers to the inlet opening.

15. The appliance as claimed in claim 2, further comprising a partitioning device for partitioning the incoming exhaled air and, optionally, for delivering a portion to a measuring chamber of the plurality of measuring chambers.

16. The appliance as claimed in claim 1, wherein the inlet opening is a mouthpiece.
